(19) United States
(12) Patent Application Publication
(10) Pub. No.: US 2025/0095846 A1
(43) Pub. Date: Mar. 20, 2025

(54) ERGONOMIC EYEWEAR TECH ENHACEMENT ACCESSORIES

(71) Applicant: WILLIS DENNIS GRAJALES, LITTLE ELM, TX (US)

(72) Inventor: WILLIS DENNIS GRAJALES, LITTLE ELM, TX (US)

(73) Assignee: WILLIS DENNIS GRAJALES, LITTLE ELM, TX (US)

(21) Appl. No.: 18/963,687

(22) Filed: Nov. 28, 2024

Related U.S. Application Data
(63) Continuation-in-part of application No. 17/468,642, filed on Sep. 7, 2021.
(60) Provisional application No. 63/075,827, filed on Sep. 9, 2020.

(51) Int. Cl. G16H 40/67; A61B 5/00; G06Q 20/32; G16H 10/60; G16H 80/00; H04B 5/79

(52) U.S. Cl. CPC G16H 40/67; A61B 5/6803; G06Q 20/3223; G16H 10/60; G16H 80/00; H04B 5/79

(57) ABSTRACT

The present disclosure pertains to an enhancement apparatus for eyewear frames, featuring a shell or housing designed to improve comfort, adjustability, and functionality through advanced Near Field Communication (NFC) technology. The apparatus material is engineered to minimize interference, and the NFC modules are positioned at a specific distance from the frame to ensure optimal functionality without interaction-related issues. The apparatus securely attaches to specific frame parts, enhancing stability and user comfort. It can be scanned using external NFC-enabled devices and accommodates various NFC chips for functionalities such as prescription data retrieval, lost-and-found tracking, biometric data collection, and eyewear programming. Depending on the sophistication of the NFC chip, it supports the storage and retrieval of diverse data types, including medical records, NFT information, and other personalized content. This innovation merges a secure, ergonomic design with seamless data management and communication, enhancing the overall functionality and user experience of eyewear.

### RELATED APPLICATION

This application is a continuation of U.S. Provisional Patent Application No. 63/075,827, filed on Sep. 9, 2020, which claims priority to U.S. Provisional patent application Ser. No. 17/468,642, filed on Sep. 7, 2021, which itself claims priority to provisional patent application No. 63/530,383 which itself claims priority to U.S. patent application Ser. No. 18/731,306, filed on Jun. 6, 2024. The disclosures of the aforementioned applications are hereby incorporated by reference in their entirety.

### FIELD OF THE INVENTION

The present invention relates to the field of wearable eyewear accessories, specifically to an enhancement apparatus for eyewear frames that integrates advanced functionalities such as Near Field Communication (NFC) technology for data retrieval, biometric data collection, and improved user interaction. The invention further pertains to adaptive and ergonomic designs that enhance the comfort, adjustability, and functionality of eyewear while enabling seamless data management and communication capabilities.

### BACKGROUND OF THE INVENTION

Traditional eyewear, while essential for vision correction, often fails to address critical user challenges related to comfort, stability, and functionality. Users commonly experience discomfort due to uneven weight distribution, leading to pressure points on the nose and ears, especially during prolonged wear. Frames frequently slip off the face during physical activity or in humid conditions, causing inconvenience and potential damage to the eyewear.

Moreover, patients who use multiple pairs of glasses for different purposes—such as reading, computer use, or distance vision—often struggle to identify which pair is suited for a particular activity. This confusion can result in improper use of eyewear, leading to eye strain, frustration, or even accidents. Current eyewear accessories do not provide an effective solution to help users differentiate between their glasses or offer smart reminders about their intended use.

Eyewear loss is another pervasive issue, as traditional glasses and sunglasses lack any form of identification or lost-and-found mechanism. Users frequently misplace their eyewear, and the chances of recovering it are slim without a way for the finder to identify the rightful owner. Similarly, there is no mechanism to deter theft or assist in recovering stolen eyewear. These challenges often leave users frustrated and lead to costly replacements.

While advanced technologies like smart glasses have become prevalent in other industries, integrating them into traditional framed eyewear presents design challenges, such as interference from metal frames and the lack of compact, adaptable solutions. The bulkiness and uneven weight distribution of smart glasses can lead to discomfort for some users, and variations in face shapes often result in poor fit, particularly regarding factors like pantoscopic tilt or bridge distance. Additionally, current eyewear lacks features that enable users to interact with digital devices or access valuable information about their framed eyewear, such as prescription details or usage guidelines.

The integration of advanced technologies into traditional framed eyewear poses several significant challenges, particularly in terms of design, comfort, and functionality. One major problem lies in the inherent bulkiness and weight distribution of smart glasses, which are often heavier than regular eyewear due to the added electronic components. This additional weight can cause discomfort, particularly for extended periods of wear, as it may create pressure points on the nose or ears. Furthermore, the weight distribution may be uneven, leading to glasses that slip or sit awkwardly on the face, making them impractical for many users.

Unlike traditional glasses, which are carefully designed to be lightweight and comfortable, smart glasses struggle to maintain the same level of user comfort. The varied shapes and dimensions of individuals' faces exacerbate this problem—factors like pantoscopic tilt, bridge distance, and overall facial structure significantly impact how well the glasses fit. Because every person has a unique facial anatomy, a one-size-fits-all approach to smart glasses often results in poor fit and reduced comfort, making them unsuitable for many potential users.

Another key challenge is the interference caused by the materials commonly used in traditional frames. Many eyeglass frames are made of metal, which can interfere with the antennas and sensors used in advanced technologies like Near Field Communication (NFC) and biometric sensors. This interference can limit the effectiveness of the smart features integrated into the eyewear, reducing their ability to perform essential functions like communication, data transmission, or sensing. Designers face a difficult task in trying to integrate these advanced technologies without compromising their functionality or aesthetics. The use of metal, while popular for its durability and sleek appearance, introduces technical barriers that make seamless integration of smart components difficult without impacting performance.

Additionally, existing traditional eyewear lacks features that enable users to interact with digital devices or access valuable information about their glasses. Users are unable to retrieve important information such as prescription details, maintenance reminders, or usage recommendations directly from their eyewear. In a world where digital interactivity is becoming increasingly common, eyewear has not kept pace with other consumer electronics in terms of convenience and accessibility. The absence of digital interactivity means that users must rely on manual methods to keep track of their prescription history or consult their eye care provider for information that could otherwise be conveniently accessible.

These challenges collectively contribute to the limited adoption of smart eyewear technology. The issues surrounding privacy, comfort, fit, and technical integration hinder the widespread acceptance of such devices. Users expect their personal accessories to be functional, comfortable, and easy to use, but if the eyewear does not fit well or feels uncomfortable, it becomes an obstacle to adoption and comfort. Moreover, without practical solutions that address the interference from frame materials or enhance digital interactivity, traditional framed eyewear cannot effectively integrate the full range of advanced technologies.

These limitations prevents evolution of current technologies for many users from experiencing the potential benefits, such as health monitoring, contactless payments, and seamless connectivity with digital devices, ultimately slowing down the growth and adoption of smart eyewear in the market.

The disclosed apparatus addresses these issues by combining ergonomic design, technological integration, and user-centric functionality into a single eyewear accessory. The invention incorporates a lightweight, flexible shell made of materials such as silicone, rubber, or plastic, which evenly distribute weight to alleviate pressure points and enhance user comfort. Strategically placed pressure points and secure attachment mechanisms, including clip-on, cavity, or magnetic options, prevent slippage and ensure stable positioning during wear.

For example, many virtual reality (VR) headsets that have entered the market, including those from prominent brands, have faced significant issues related to weight distribution. Due to the heavy front-facing components of these headsets, users often experience discomfort during extended use. The poor weight balance can lead to undue strain on the neck, ears, causing neck pain or even musculoskeletal problems over time. This is particularly problematic because the added pressure on sensitive areas, such as the neck and head, can trigger migraines or exacerbate pre-existing conditions for some users. These issues illustrate how crucial it is to get the ergonomics right when integrating advanced technologies into wearable devices.

Smart eyewear faces similar challenges, as the uneven weight distribution and bulk of electronic components can significantly affect user comfort, ultimately deterring individuals from adopting such technology. Just like with VR headsets, ensuring that smart eyewear is comfortable, well-balanced, and suitable for a variety of facial structures is vital to overcoming the challenges of wearability and enhancing user experience.

A key feature of this invention is the integration of NFC chips technology, which allows users to interact with their glasses in meaningful ways. By tapping or scanning 35 their glasses with an NFC-enabled device, such as a smartphone, computer, smart hub or even a smart car system, users can retrieve information about the glasses. For instance, the glasses can communicate that they are designed for reading, computer use, or distance vision, helping users quickly identify the correct pair for their needs. This functionality reduces confusion, promotes proper eyewear use, and enhances convenience for patients managing multiple prescriptions.

Additionally, the apparatus introduces a robust lost-and-found mechanism for eyewear. If a person misplaces their glasses, the finder can tap the glasses with an NFC-enabled device to retrieve contact information for the rightful owner. This greatly improves the likelihood of recovering lost eyewear. In cases of theft, law enforcement can use NFC-enabled devices to scan the glasses and verify ownership, as the stored data will confirm that the glasses belong to a different individual or store. This feature provides added security and accountability, making eyewear not just a personal accessory but also a trackable and recoverable item.

The invention also overcomes technological challenges by including a dedicated compartment for NFC chips and antennas, carefully designed to prevent interference from metallic frame components. This ensures reliable communication with external devices, enabling seamless data transfer and programmability. Additional applications include telemedicine connectivity, appointment scheduling, and prescription storage, making the accessory a versatile tool for both consumer and healthcare environments.

Furthermore, the outer shell of the apparatus is customizable, allowing users to personalize their eyewear with logos, decorative elements, or ornamental designs. Certain embodiments of the apparatus may also include biometric sensors integrated into the skin-contacting portion, enabling health monitoring features such as tracking vitals or recording user data for medical purposes.

By combining enhanced comfort, stability, digital connectivity, and security, the disclosed apparatus offers a comprehensive solution to major problems faced by eyewear users. This single accessory provides multiple functionalities, transforming eyewear into an interactive, multifunctional device tailored to meet the evolving demands of modern users while addressing critical issues like comfort, usability, loss prevention, and theft deterrence.

### SUMMARY OF THE INVENTION

The disclosed invention provides a multifunctional apparatus for eyewear that enhances eyewear comfort, eyewear stability, and functionality while integrating advanced features such as Near Field Communication (NFC) technology. The apparatus includes a lightweight, flexible shell made of materials like silicone, rubber, or plastic to reduce pressure points and improve wearer comfort. Secure attachment mechanisms, including clip-on, magnetic, and cavity-based designs, prevent eyewear slippage and ensure stable positioning during use.

A key feature of the apparatus is a shell that houses an NFC module, which supports at least one of: programming, data retrieval, and data communication. This enables functionalities such as prescription retrieval, lost-and-found mechanisms, and secure interactions with external devices for tasks like healthcare monitoring, access control, and payment processing. In some embodiments, the apparatus can store and transmit user-specific data, such parts of medical records or biometric logs, while maintaining compatibility with industry standards for NFC and RFID technologies.

The apparatus may be used upgradable advanced NFC/RFID chips that supports biometric sensing capabilities in certain configurations, allowing the apparatus to monitor physiological parameters such as Glucose levels, body temperature, heart rate, or motion. These readings can trigger alerts or provide real-time updates to external devices for health tracking and wellness management.

Furthermore, the external shell of the apparatus may be designed for aesthetic customization, accommodating logos, Glow in the dark designs, color temperature changing, decorative elements, cartoons, and programmable features like LED lighting. The apparatus is adaptable to a wide range of eyewear styles, including prescription glasses, sunglasses, and reading glasses, offering a scalable solution for integrating advanced technologies into traditional eyewear designs.

This invention transforms eyewear into a versatile platform for personal, healthcare, and consumer applications, addressing critical challenges such as comfort, usability, data security, and functionality, while providing an aesthetically pleasing and user-friendly solution.

### BRIEF DESCRIPTION OF THE DRAWINGS

Embodiments of the apparatus, methods of use, wearable devices, and related kits, designs, or systems are described below, with reference to the accompanying figures, which are not drawn to scale. The figures are annotated with reference numbers representing various features and components, and these numbers are used throughout the description as an aid for better understanding, with identical numbers indicating the same or similar features and components.

FIG. 1 is a schematic diagram illustrating a system integrating Near Field Communication (NFC) technology into an eyewear apparatus 1, showing housing apparatus 1 (26), NFC sensor (27), and various functionalities such as sensor data collection (12), lost and found information (20), prescription storage (7), and payment processing (72).

FIG. 2 is a schematic diagram of an eyewear adapter device configured to facilitate payment processing through integrated NFC technology, depicting an NFC eyewear adapter (4) interacting with a payment terminal (3), thereby eliminating the need for traditional payment methods.

FIG. 3 is a schematic representation of a system and method for programming NFC or RFID tags within the apparatus casing or shell, while using a mobile application, illustrating the mobile application interface (36, 43) for data entry and programming workflow, and the role of an NFC programmer (42).

FIG. 4 illustrates an embodiment of eyewear accessories integrated with NFC tag technology, highlighting NFC tag placements (1) on various eyewear components such as the frame rim (64), bridge (65), temples (2), temple tips (67), and nose pads (66), and indicating data transmission (3) with external devices.

FIG. 5 illustrates a versatile housing solution (1) adaptable for various wearable devices, incorporating sensing and communication technologies like NFC, RFID, or hybrid sensors (27).

FIG. 6 illustrates the integration of a multifunctional sensor housing (1) into an eyewear device (4), demonstrating its use in contact or proximity to the user's skin for enhanced data collection and functionality.

FIG. 7 shows multiple views (front, bottom, and top) of a multifunctional housing or shell apparatus 1 (1) designed for integration with wearable devices, particularly framed eyewear, accommodating advanced sensing technologies while ensuring compactness and ergonomic adaptability.

FIG. 8 illustrates a programmable eyewear accessory designed for both comfort and decoration. The diagram features a “Framed Eyewear Attachment Mechanism 4,” enclosed within a shell, casing, or housing labeled as “110.” Within this enclosure, the figure depicts an “NFC TAG 140,” which represents the near-field communication component of the accessory. Additionally, an “Antenna 27” is shown, suggesting that this component is used to facilitate wireless communication for the NFC functionality. The components are labeled for easier identification, and the structure appears to be designed to attach to or integrate with eyewear frames, enhancing their utility with programmable features.

FIG. 9 depicts a programmable eyewear accessory designed for both comfort and decoration. The accessory includes various components within a “Framed Eyewear Attachment Mechanism” and a shell or housing structure. The key features are labeled, including an attach mechanism (4), an NFC chip (140), memory (130), and an antenna (27). Additionally, there is a “Sensor and/or Antenna” component (140), which suggests additional functionality for sensing or communication. Below the main attachment mechanism, there is a “Power Source” (250), which encompasses different elements for powering the device. This power system includes a battery source (252), an energy harvesting module (254), and a magnetic coupling mechanism for power transfer (252). These features suggest that the device can utilize different forms of energy, potentially combining traditional battery power with energy-harvesting capabilities for more efficient use.

The overall design aims to add programmable and interactive features to eyewear, enhancing user comfort and personalization while providing advanced connectivity options.

FIG. 10 illustrates a programmable eyewear accessory designed for both comfort and decoration, emphasizing its attachment and connectivity features. The diagram presents several key components housed within a “Shell, Casing, or Housing” (110). These components include an “Attach Mechanism” (4), an “NFC TAG” (140), a “Sensor” (140), an “Antenna” (27), and “Memory” (130) for storing programmable data (38). The attach mechanism (4) offers different options for securing the accessory to the eyewear, such as magnetic attachments, snap-on clips, adhesive or suction attachments, and cavity-based slip-fit mechanisms, allowing for versatility and ease of use. The presence of an NFC tag, along with an antenna and a sensor, indicates that the accessory supports wireless communication and sensing capabilities, potentially for interactions with other devices. The programmable data stored in memory (38) further suggests customization possibilities for the user's experience.

FIG. 11 is presenting a programmable eyewear accessory for comfort and decoration, specifically showcasing a “Sensor Module” (120) integrated with various health monitoring capabilities. The module includes several sensors capable of measuring different physiological parameters. These include a pH sensor (450), a glucose sensor (440), a temperature sensor (430), an oxygen saturation sensor, often referred to as a pulse oximeter (420), and a heart rate sensor (420). Additionally, an antenna (27) is included as part of the sensor module, and there are options for NFC/RFID chips, indicating that the accessory can wirelessly communicate data. The health sensors integrated into the eyewear suggest that the accessory has potential applications for real-time health monitoring, offering convenience and non-intrusive data collection for the user. This makes the eyewear accessory versatile, combining both decorative elements and practical health tracking features, suitable for those who want enhanced functionality in their eyewear.

FIG. 12 is a block diagram further illustrating depicts a programmable eyewear accessory designed for both comfort and decoration, showcasing several key components integrated within an attachment mechanism and a protective housing or casing (110). The diagram highlights “NFC Tech” (140), which indicates the use of near-field communication technology for connectivity purposes. A “Sensor Module” (120) is also included, containing an antenna (27) and memory (130). The antenna serves as the communication link, enabling data exchange, while the memory module stores information, possibly related to user preferences or programmed features. These components are housed within a compact structure that can be attached to eyewear, providing a combination of functionality, such as wireless communication and health tracking, while also enhancing the aesthetic appeal of the eyewear. This design makes it versatile for users who want added features beyond standard eyewear, emphasizing both practicality and personalization

FIG. 13 is a block diagram further illustrates a programmable eyewear accessory for comfort and decoration, featuring components integrated within a “Framed Eyewear Attachment” and a protective housing or casing (110). The key elements include a memory unit (620) that stores different types of data, enhancing the functionality of the accessory. Specifically, the memory holds medical data (38), eyeglass prescription information (624), and web link data (12), suggesting that this accessory can provide various forms of personalized information, including health-related records and direct access to online resources.

FIG. 14 is a flowchart illustrating the process of using a programmable eyewear accessory for comfort and decoration. The process starts with attaching a casing that contains a sensor module, a memory module, and an NFC module to the eyewear frames (step 710). Once attached, the sensor module is configured to detect one or more biometric parameters from the user (step 720). These biometric parameters, along with any user-specific data, are then stored in the memory module (step 730). The final step in the process involves transmitting the stored data to an external processor via the NFC module (step 740). The flowchart illustrates a streamlined workflow for gathering biometric information and enabling communication between the eyewear accessory and external systems, offering a seamless way to integrate health monitoring features with wearable technology.

### DETAILED DESCRIPTION

The following detailed description is provided to enable those skilled in the art to make and use the invention and is not intended to limit the scope of the invention. The invention comprises an eyewear wearable accessory device, integrated systems, and methods utilizing Near Field Communication (NFC), Radio Frequency Identification (RFID), and/or hybrid sensor technologies for multifaceted applications, including data exchange, bio-sensing 12, payment processing 72, comfort and medical use. Component 4 serves as an adapter mechanism for connecting, embedding, or adapting the apparatus 1 to framed eyewear.

In some embodiments, the present disclosure relates to an Apparatus 1, an accessory for framed eyewear that integrates ergonomic design, advanced technological functionality, and user-centric features to address longstanding issues with traditional eyewear, including discomfort, instability, lack of differentiation for multiple prescriptions, and challenges associated with loss or theft 20.

In some embodiments, the Apparatus 1 includes a lightweight, flexible shell fabricated from at least one materials such as silicone, epoxy, rubber, or plastic 33. These materials are chosen for their durability, flexibility, and ability to evenly distribute weight across the user's facial structure, thereby alleviating pressure points on the nose and ears. This ergonomic design significantly reduces user discomfort, particularly during extended periods of wear.

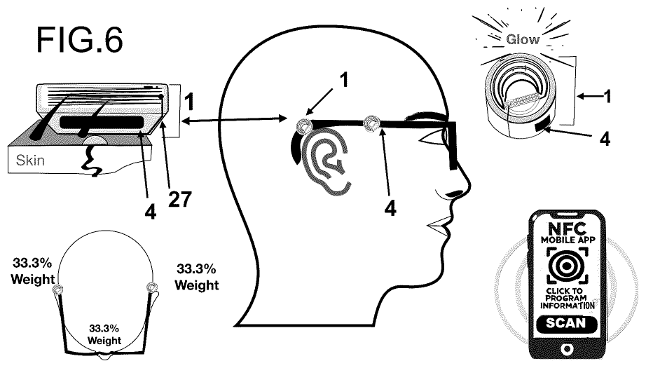

In some embodiments, the Apparatus 1 includes a lightweight, flexible shell 4 fabricated from materials such as silicone, rubber, or plastic. These materials are chosen for their durability, flexibility, and ability to evenly distribute weight across the user's facial structure, thereby alleviating pressure points on the nose and ears (FIG. 6). This ergonomic design significantly reduces user discomfort, particularly during extended periods of wear.

In some embodiments, to ensure stability during routine and active use, Apparatus 1 incorporates secure attachment mechanisms 4. These mechanisms include, but are not limited to, clip-on designs 33, magnetic fasteners 4, or cavity-based grips 4. They prevent slippage even under conditions of high humidity or during vigorous physical activity. The modular outer shell 4 is adaptable to a variety of eyewear frame shapes and sizes, enhancing compatibility. Additionally, Apparatus 1 allows for aesthetic customization, enabling integration of logos, decorative patterns, or programmable LED lighting, thus providing both functional and aesthetic benefits.

In some embodiments, a key feature of Apparatus 1 is the incorporation of Near Field Communication (NFC) technology. The NFC module is embedded within a dedicated compartment in the shell or frame, designed to minimize interference from metallic frame components and ensure reliable operation.

In some embodiments, a key feature of Apparatus 1 is the incorporation of (RFID) technology. The RFID or NFC 27 module is embedded within a dedicated compartment in the shell or frame, designed to minimize interference from metallic frame components and ensure reliable operation.

In some embodiments, the NFC 27 functionality provides multiple user benefits, including the storage and retrieval of prescription information. By tapping 35 an NFC-enabled device, such as a smartphone, against Apparatus 1, users can access data such as the prescription type (e.g., reading, distance, or computer use), intended functionality, and usage recommendations. This feature is particularly advantageous for users managing multiple pairs of glasses, reducing confusion and promoting proper eyewear use.

In some embodiments, in addition, the NFC module in Apparatus 1 facilitates a lost-and-found mechanism. If eyewear is misplaced, a finder can tap the accessory with an NFC-enabled device to retrieve contact information for the rightful owner, significantly increasing the likelihood of recovery. In cases of theft, law enforcement or authorized personnel can scan the NFC module to verify ownership through stored identification data, thereby enhancing security and aiding in recovery efforts.

In certain embodiments of Apparatus 1 incorporate biometric sensors into the skin-contacting portions of the accessory. These sensors are capable of monitoring physiological parameters such as heart rate, body temperature, and motion (via accelerometers or gyroscopes). Data collected by these sensors can be transmitted to external devices in real time, enabling advanced health monitoring. For instance, the biometric sensors in Apparatus 1 can support chronic disease management, fitness tracking, and emergency response by generating alerts for abnormal readings or notifying healthcare providers of critical conditions.

In some embodiments, the Apparatus 1 may further include Radio Frequency Identification (RFID) technology to complement NFC functionality or hybrid system. The RFID system allows the accessory to operate as both a tag and a reader. Applications of the RFID module include inventory tracking in retail environments, secure user identification for access systems, and dynamic location monitoring in professional or medical settings. This dual capability enhances the versatility of Apparatus 1 for both consumer and enterprise applications.

In some embodiments, the Apparatus I may be powered by a rechargeable lithium-ion battery housed within the shell or frame. The battery supports extended operation and can be recharged via multiple methods, including solar charging and Qi wireless charging. The NFC and RFID modules are shielded within dedicated compartments to prevent electromagnetic interference, ensuring reliable communication even in the presence of metallic frame components.

In other embodiments, the Apparatus 1 addresses a wide range of applications across personal, professional, and medical domains. In healthcare, the accessory enables real-time health monitoring, telemedicine connectivity, and management of chronic conditions such as diabetes or cardiovascular diseases. In retail environments, the NFC and RFID functionalities support contactless payments, inventory tracking, and customer engagement. For professional use, Apparatus 1 facilitates secure access control and employee wellness tracking.

In other embodiments, in personal use cases, the Apparatus 1 simplifies the user's interaction with their eyewear by enabling immediate access to prescription details and improving the recovery of lost or stolen items. Furthermore, the customizable outer shell ensures that the accessory meets the aesthetic and functional preferences of a diverse user base.

In other embodiments, challenges such as locating misplaced eyewear in low-light environments remain unaddressed. To overcome this limitation, optional glow-in-the-dark designs the apparatus offer a practical solution by making eyewear visible in conditions such as nighttime at a beach. Additionally, the need for aesthetic customization is increasingly important, with users seeking eyewear that blends advanced functionality with visual appeal.

In some embodiments, the present apparatus addresses these gaps by providing an enhancement apparatus designed for attachment to eyewear frames with additional elements. This apparatus combines advanced NFC technology with features that may include glow-in-the-dark elements, customizable designs, and programmable capabilities, transforming any eyewear into a versatile platform for personal, healthcare, and consumer applications.

In other embodiments, the Apparatus 1 effectively addresses critical challenges associated with traditional eyewear, such as discomfort, instability, confusion between multiple prescriptions, and the absence of recovery mechanisms for lost or stolen glasses. By integrating ergonomic design with advanced NFC, RFID, and biometric sensing technologies, Apparatus 1 transforms eyewear into an interactive, multifunctional device. This innovation enhances user experience while providing a scalable solution adaptable to evolving technological standards and consumer demands. Modifications and enhancements to the described embodiments are anticipated to ensure broad applicability and alignment with future user needs.

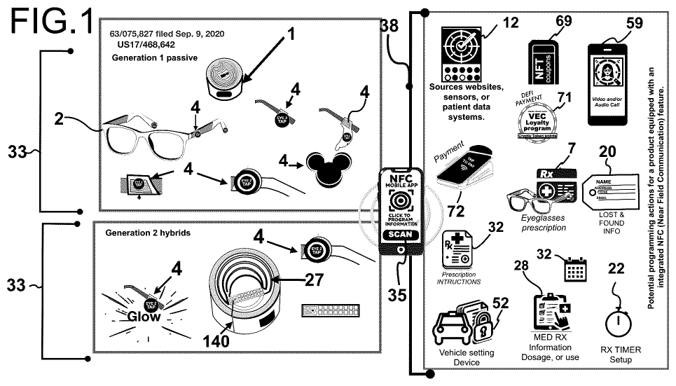

In some embodiments, FIG. 1 illustrates the functionalities and potential programming actions for a Programmable Eyewear Accessory equipped with NFC (Near Field Communication) technology. The figure is divided into two sections: Generation 1 Passive and Generation 2 Hybrids, showcasing the evolution of the accessory. In the Generation 1 Passive section, the accessory includes NFC-enabled modules (Eye2Tap 4) that can be attached to eyewear for functionalities such as data access, decorative customization (e.g., bunny charm), and storage of essential user information. The Generation 2 Hybrids section highlights advanced features, integrating Antenna 27 and enhanced NFC modules (Eye2Tap 140) for expanded functionalities, including data communication and sensing capabilities.

In some embodiments of FIG. 1, the right side of the figure details the diverse applications enabled by the accessory. These include storing Web, Sensor, or Patient Data 12, facilitating Video/Audio Calls 59, managing NFT Coupons 69, supporting Payment 72, and enabling loyalty programs like VEC Crypto Token Points 71. Additionally, the device can store Eyeglass Prescription Information 7, Lost and Found Info 20, Prescription Instructions 32, and Medication Dosage or Usage Information 28. It also offers features like Vehicle Settings 52 and RX Timer Setup 22, showcasing its multifunctionality. This figure demonstrates the accessory's ability to integrate NFC technology into eyewear, enabling a wide range of use cases such as medical data management, payment systems, digital loyalty programs, and interactive features, while emphasizing its potential for decorative and functional enhancements.

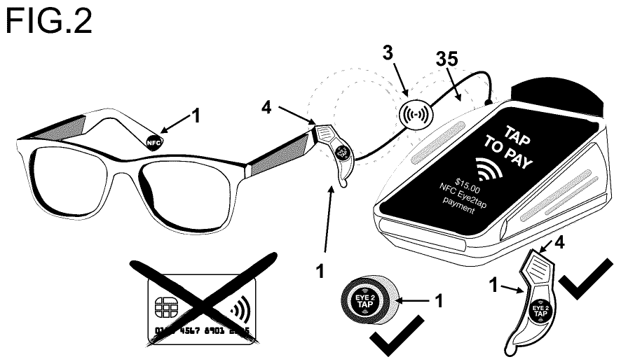

In some embodiments, the FIG. 2 provides a schematic illustration of an eyewear device configured for payment processing through integrated NFC technology. The adapter mechanism (4) plays a critical role in embedding the NFC-enabled housing unit into the eyewear. Eyewear Frame (1): Incorporates an NFC-enabled housing unit connected via the adapter mechanism (4), designed to facilitate tap-to-pay functionality with external payment terminals (3). Eye2Tap Adapter (4): A detachable or embedded module positioned on the temple of the eyewear frame. It enables seamless integration of the NFC payment system while maintaining the form factor of the frame. Payment Terminal (3): Depicts the transaction process with a contactless payment system, confirming successful integration and operation. In some embodiments, the adapter (4) ensures a secure and modular attachment, allowing advanced functionality while preserving the physical integrity and comfort of the eyewear.

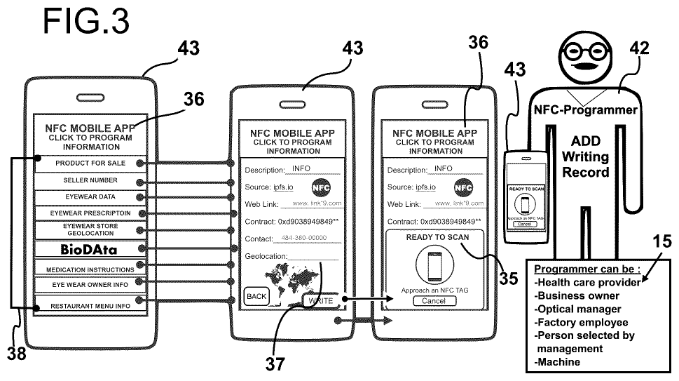

In some embodiments, FIG. 3 illustrates the process of programming NFC tags using an NFC Mobile App interface and highlights the roles of an NFC Programmer 42. The figure displays a sequence of mobile screens (36) where users can input and program information such as product details, seller numbers, eyewear data, eyewear prescriptions, geolocation, medication instructions, and even restaurant menu info. The app allows users to write the data to an NFC tag by selecting relevant details and pressing the Write Button 37, after which the tag is ready to scan.

In some embodiments, the NFC Programmer 42 represents individuals responsible for managing the programming process. These can include healthcare providers, business owners, optical managers, factory employees, users, owners or other personnel designated by management. The final screen (35) confirms readiness to scan and complete the programming process. This figure demonstrates the accessory's functionality in facilitating customizable and secure data programming for various applications using NFC technology.

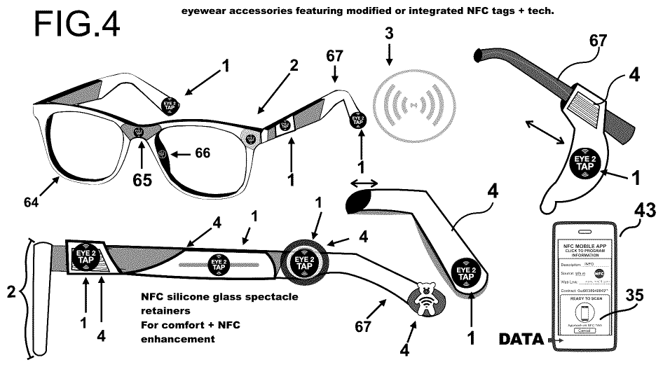

In some embodiments, FIG. 4 illustrates eyewear accessories featuring modified or integrated NFC tags and technology for enhanced functionality and user comfort. The eyewear includes Eye2Tap modules 1, strategically embedded in various parts of the frame, such as the temples 2, nose pads 66, and bridge 65, enabling seamless NFC communication. The accessory integrates silicone glass spectacle retainers, ensuring a comfortable fit while supporting NFC functionalities. Additional features include temple tips 67, which may host decorative elements or functional NFC modules.

In some embodiments, the figure also demonstrates how the NFC-enhanced framed eyewear interacts with an NFC Mobile App, allowing users to program and retrieve information like product details, eyewear prescriptions, or other digital data. The Frame Rim 64 and Lens 63 maintain the accessory's core structural integrity while incorporating advanced features. This design highlights the integration of NFC technology to provide data management, user customization, and aesthetic enhancement in a practical and wearable format.

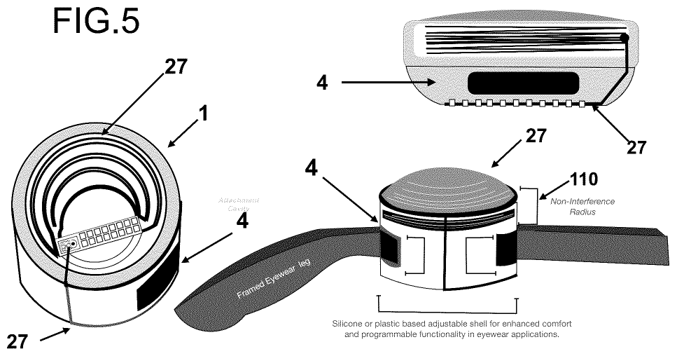

In some embodiments, FIG. 5 depicts a Programmable Eyewear Accessory designed for comfort, adjustability, and programmability. The accessory integrates a silicone shell 1, which enhances the comfort of eyewear while providing a secure fit around the framed eyewear leg 4. The shell is specifically engineered to maintain a non-interference distance, ensuring that the integrated NFC and other communication technologies remain unaffected by surrounding materials. The accessory features an Antenna 27, embedded within the shell, to enable efficient data transmission and connectivity. The detailed views highlight the modular structure of the shell, emphasizing its adaptability to various eyewear designs. This figure showcases the accessory's ability to combine user comfort with advanced technology, ensuring seamless integration and functionality for modern eyewear applications.

In some embodiments, FIG. 6 illustrates the application of a Programmable Eyewear Accessory featuring a silicone shell 1 and attachment mechanism 4, highlighting its placement and interaction with the user's skin. The accessory is securely attached to the temple of the eyewear frame, ensuring a stable and comfortable fit around the head. The Antenna 27, embedded within the silicone shell, is positioned to maintain effective communication while avoiding interference with the user's skin or surrounding materials. The design ensures that the accessory provides programmability and functionality, such as biometric data collection or NFC-based features, without compromising user comfort. This figure emphasizes the ergonomic integration of advanced technologies into eyewear for seamless daily use.

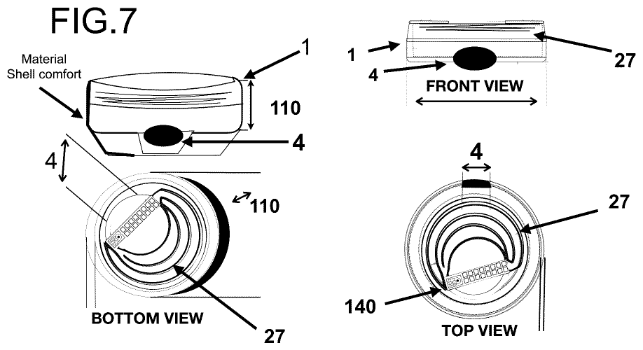

In some embodiments, FIG. 6 illustrates the application of a Programmable Eyewear Accessory featuring a silicone shell 1 and attachment mechanism 4, highlighting its placement and interaction with the user's skin. The accessory is securely attached to the temple of the eyewear frame, ensuring a stable and comfortable fit around the head. The Antenna 27, embedded within the silicone shell, is positioned to maintain effective communication while avoiding interference with the user's skin or surrounding materials. The design ensures that the accessory provides programmability and functionality, such as biometric data collection or FIG. 7 provides detailed views of a Programmable Eyewear Accessory focusing on its structural design and functional components. The Front View highlights the silicone material shell 1, which is designed for user comfort and provides a secure fit when attached to eyewear via the attachment mechanism 4. The design ensures a non-interference distance, maintaining the integrity of embedded components like the Antenna 27 for optimal data transmission and communication.

In some embodiments, FIG. 6 includes Top View showcases the placement of the NFC module 140 within the shell, emphasizing its integration with the overall design to enable programmable and customizable functionalities. The Bottom View illustrates the internal layout, including the Antenna 27 and other components, designed to optimize performance while ensuring ergonomic comfort for the user. These views collectively demonstrate the accessory's ability to combine comfort, secure attachment, and advanced technological capabilities in a compact and efficient form factor.

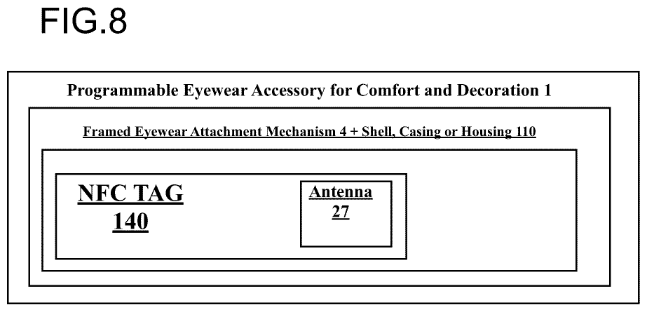

In some embodiments, FIG. 8 illustrates a Programmable Eyewear Accessory for Comfort and Decoration 1, featuring a Framed Eyewear Attachment Mechanism 4 integrated within a Shell, Casing, or Housing 110. The accessory includes an NFC Tag 140, enabling short-range communication for various programmable functions, and an Antenna 27, which facilitates efficient data transmission and connectivity. This compact and modular design allows for seamless integration into eyewear frames, offering enhanced functionality while maintaining user comfort and practicality.

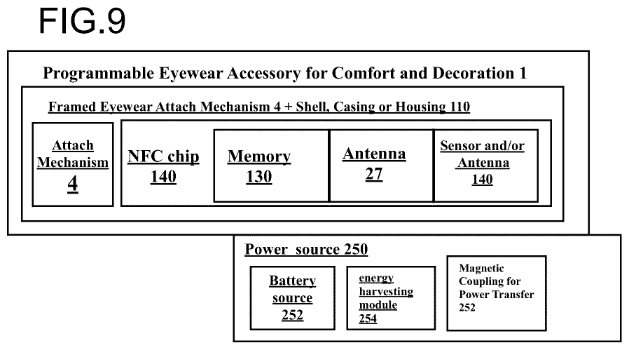

In some embodiments, FIG. 9 illustrates a Programmable Eyewear Accessory for Comfort, Iot and Decoration 1, showcasing its components and seamless functional integration. The accessory features a Framed Eyewear Attach Mechanism 4 paired with a Shell, Casing, or Housing 110, ensuring secure attachment to eyewear frames. It incorporates an NFC Chip 140 for short-range communication, a Memory 130 for storing data, and an Antenna 27 to enable data transmission and maintain connectivity. Additionally, the design includes a Sensor and/or Antenna Module 140, offering advanced functionality such as environmental monitoring or biometric sensing. The accessory is powered by a versatile Power Source 250, which includes multiple energy options: a Battery Source 252 for direct power, an Energy Harvesting Module 254 to harness environmental energy like solar or kinetic sources, and Magnetic Coupling for Power Transfer 252 for wireless charging via magnetic induction. This innovative design integrates data communication, sensing technologies, and efficient energy management into a compact and wearable format, providing both functionality and comfort tailored specifically for eyewear.

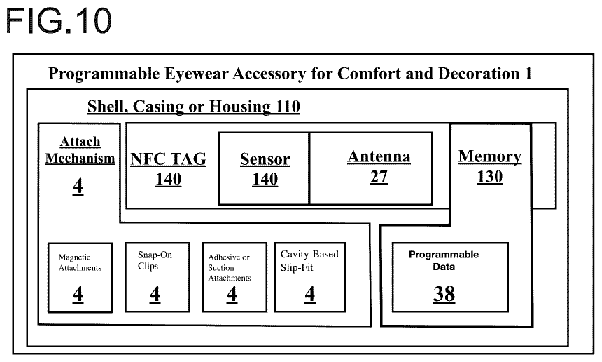

In some embodiments, FIG. 10 illustrates a Programmable Eyewear Accessory for Comfort and Decoration 1, showcasing the core components enclosed within a Shell, Casing, or Housing 110. The accessory includes an Attach Mechanism 4, which offers multiple options such as magnetic attachments, snap-on clips, adhesive or suction attachments, and cavity-based slip-fit designs, ensuring compatibility with various eyewear frames. The NFC Tag 140 enables short-range communication for programmable functionalities, while the Sensor 140 gathers environmental or biometric data. The Antenna 27 facilitates data transmission, ensuring seamless connectivity, and the Memory 130 stores Programmable Data 38, which may include user-specific or application-specific information. This figure highlights the accessory's capability to combine secure attachment mechanisms with advanced technology, enhancing both comfort and functional integration for eyewear.

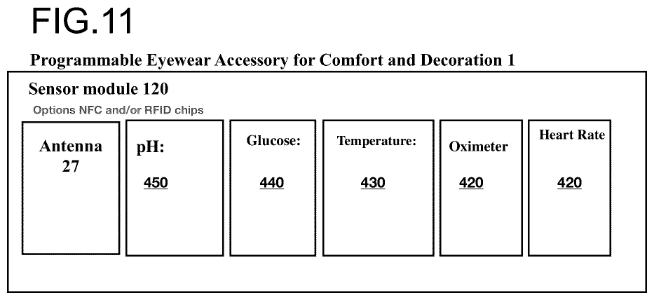

In some embodiments, FIG. 11 illustrates a Programmable Eyewear Accessory for Comfort and Decoration 1 with a Sensor Module 120 equipped to provide multiple biometric and environmental monitoring capabilities. The module integrates an Antenna 27, enabling communication through NFC/RFID chips, and various sensor functionalities. These sensors include a pH Sensor 450 for measuring skin or environmental acidity, a Glucose Sensor 440 for potential non-invasive glucose monitoring, a Temperature Sensor 430 for capturing body or ambient temperature, an Oxygen Saturation Sensor (Pulse Oximeter) 420 for monitoring blood oxygen levels, and a Heart Rate Sensor 420 to measure the user's pulse. This figure highlights the accessory's capability to combine advanced biometric sensing with communication technologies, offering a comprehensive wearable solution for health monitoring and data transmission.

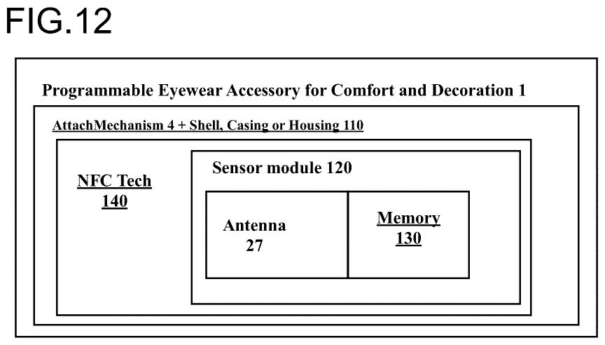

In some embodiments, FIG. 12 depicts a Programmable Eyewear Accessory for Comfort and Decoration 1, which integrates an Attach Mechanism 4 along with a Shell, Casing, or Housing 110 to secure the accessory onto eyewear frames. The core components include an NFC Technology Module 140, enabling short-range communication for various applications, and a Sensor Module 120, which houses an Antenna 27 and a Memory 130. The Antenna 27 facilitates data transmission and reception, while the Memory 130 stores relevant data, such as programming configurations, user-specific information, or application-related data. This figure demonstrates how the accessory combines advanced communication technologies with a modular design to enhance the functionality, usability, and comfort of eyewear.

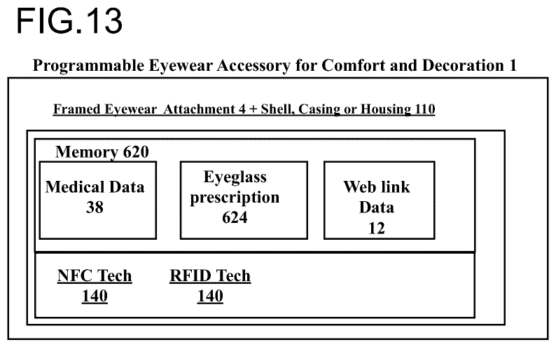

In some embodiments, FIG. 13 illustrates a Programmable Eyewear Accessory for Comfort and Decoration 1, showcasing a Framed Eyewear Attachment integrated with a Shell, Casing, or Housing 110. The accessory includes a Memory 620 component designed to store various types of user-specific data, such as Medical Data 38 (health records or biometric information), Eyeglass Prescription 624 (digital storage of prescriptions for easy access), and Web Link Data 12 (URLs or links to related digital content or services). Additionally, the accessory incorporates NFC Technology 140, which enables secure short-range communication for sharing or retrieving stored data, and RFID Technology 140, which facilitates long-range communication for extended functionality. This figure highlights the integration of advanced memory and communication technologies into a wearable accessory, providing both practical data management and enhanced user comfort while maintaining aesthetic appeal.

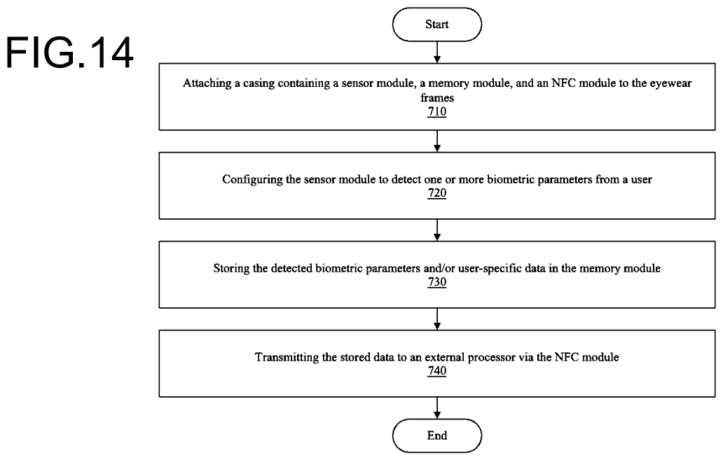

In some embodiments, FIG. 14 is a flowchart that describes a method for integrating a framed eyewear wearable device, according to some embodiments of the present disclosure. In some embodiments, at 710, the method may include attaching a casing containing a sensor module, a memory module, and an NFC module to the eyewear frames. At 720, the method may include configuring the sensor module to detect one or more biometric parameters from a user. At 730, the method may include storing the detected biometric parameters and/or user-specific data in the memory module. At 740, the method may include transmitting the stored data to an external processor via the NFC module.

In some embodiments, the disclosed apparatus functions as a multifunctional accessory for eyewear, providing enhanced comfort, stability, and additional functionality. The device is designed to reduce tension caused by the weight of eyeglasses or sunglasses in specific areas when positioned on the user, thereby improving comfort and preventing the eyewear frame from slipping off the face. The apparatus includes a shell made from flexible, user-friendly materials such as silicone, plastic, or rubber, ensuring a comfortable fit without causing discomfort to the wearer.

In some embodiments, the shell also incorporates pressure points strategically placed to alleviate discomfort and improve the user's overall experience. Within the shell, the apparatus houses a top or outer section where NFC chips or antennas are positioned. These components are spaced appropriately to prevent interference from metallic elements in the eyewear frame, enabling seamless communication with external NFC devices. This spacing design is critical for ensuring effective signal transmission and maintaining functionality.

In some embodiments, the central compartment of the apparatus may include an attachment mechanism, which may vary depending on the design. Examples include clip-on attachments, cavity mechanisms allowing the frame's temple to slide through, or magnetic connections. The bottom section of the apparatus, which comes into contact with or is positioned near the user's skin, is designed to provide additional comfort.

In some embodiments, the external surface of the shell may serve decorative purposes, accommodating logos, cartoons, 3 dimensional designs, or other ornamental designs to enhance the aesthetic appeal of the eyewear. In certain embodiments, the apparatus may also include sensors integrated into the skin-contacting portion of the shell for biometric data collection. These sensors can provide functionality for health monitoring or other applications.

In some embodiments, the NFC component of the apparatus supports at least one or more of: a passive mode and active modes. It can be used to program or retrieve data when tapped with an external NFC-enabled device or act as an intermediary for communication with other devices. This programmable IoT eyewear accessory is engineered to combine aesthetic enhancements, superior comfort, and advanced technological capabilities to meet diverse user needs.

In some embodiments, the apparatus 1 for eyewear frames, may incorporate modular parts for NFC chips or attachment systems that can be tailored to specific functionalities based on the chip's configuration and intended use. The apparatus 1 may be designed to use various type of NFC or combination of NFC technology either collect biometric data or function as a programming system for eyewear, depending on the complexity of the chip used. For example, passive chips may enable the retrieval of programmed data from the apparatus 1 with medical information, NFT data, or other user-specific details.

In some embodiments, the NFC chip within the apparatus 1 may be strategically positioned relative to the eyewear frame material to prevent interference while optimizing functionality. In some configurations, a portion of the chip may be designed to come into direct contact with the user's skin, or be placed near the skin, depending on the intended application.

In some embodiments, where the apparatus may be called Eye2Tap™. The apparatus 1 may be utilized for biometric glucose monitoring 12, the module is positioned on the eyewear frame 2 such that the top surface is oriented away from the user, while the bottom surface maintains contact with the user's skin. This configuration enables accurate collection of physiological data 12 while securely attaching to the eyewear frame. Alternatively, in embodiments where the Eye2Tap™ adapter 1 is designed with no biometrical sensors 120, such as payment processing uses, the apparatus 1 may exclude additional sensors or position them in a manner that does not necessitate direct skin contact for operation.

This embodiment enhances the functionality of eyewear frames 2, adding capabilities that would not be achievable without the apparatus 1. By integrating apparatus 1 with its silicone shell structure 110, NFC, biometric sensing 140, secure communication, and data programming features, the apparatus 1 transforms traditional eyewear 2 into a multifunctional platform for healthcare, financial transactions, and personalized user experiences.

In some embodiments, the apparatus 1 is designed to function as a passive NFC device, attachable 140 to a section of framed eyewear and positioned at a distance sufficient to avoid interference from surrounding materials using one or more materials. It derives power solely from an external NFC reader during communication.

In some embodiments, alternative designs include the apparatus 1 that incorporates an integrated at least one or more of: active power source, passive source 252, a rechargeable battery 252 or a capacitor 252, to support extended functionalities beyond NFC communication limitations (FIG. 9).

In some embodiments, the apparatus 1 may be a hybrid RFID/NFC device configured to use passive RFID for long-range communication and use active NFC for secure, short-range data transfer and programming.

In some embodiments, the apparatus 1 may be hybrid RFID/NFC device configured other forms of wireless communication such as cellular wireless network, Bluetooth 35, ZigBee 35, or Wi-Fi 35.

In some embodiments, the apparatus 1 may be configured to operate in hybrid power mode, combining energy harvesting from NFC communication with a supplementary external active power source.

In some embodiments, the apparatus 1 may support an NFC module enhancing its communication protocols, including Bluetooth Low Energy (BLE) or Wi-Fi, for transmitting data to external devices.

In some embodiments, the apparatus 1 may be compatible with ISO/IEC 14443 and ISO/IEC 15693 standards for NFC and ISO/IEC 18000 standards for RFID. In some embodiments, the NFC module may be configured to store and transmit user-specific data, including eyeglass prescriptions or medical records, and operate without a dedicated power source when connected to an external NFC reader (FIG. 3).

In some embodiments, the casing may be configured for attachment 4 to the temple arm or bridge of eyewear frames using at least one or more of: clip-on, magnetic, cavity or adhesive coupling mechanism (FIG. 4). In some embodiments, the apparatus 1 may be configured for use with prescription glasses 2, sunglasses 2, or other optical eyewear. In some embodiments, the sensor module may include a thermistor for detecting body temperature, a photoplethysmogram (PPG) sensor for detecting heart rate, and an accelerometer for motion detection.

In some embodiments, the memory module may be configured indirectly or directly retrieve or store at least one of medical records 27, eyeglass prescriptions, or user-specific preferences, including customized settings for eyewear adjustments. In some embodiments, the apparatus 1 may be configured to provide real-time notifications of biometric parameter deviations, such as abnormal heart rate or body temperature, to an external device.

In some embodiments, the sensor module may be positioned on the temple arm or bridge of the eyewear frame in direct contact with the skin of the user (FIG. 6). In some embodiments, the memory module may be configured to store a log of biometric data for a pre-set duration, and the NFC module transmits the log to an external device upon request. In some embodiments, the NFC module enables secure access control, allowing the user to unlock doors, vehicles, or secure locations by tapping 35 the apparatus 1 near an NFC-enabled reader.

In some embodiments, the Eye2Tap apparatus 1 may be configured to interact with an external healthcare system to provide remote monitoring of biometric data 120, including heart rate 420 and body temperature 430 (FIG. 11). In some embodiments, the NFC module facilitates access control by enabling the user to unlock doors or vehicles 52 by tapping (methodology action) 35 the apparatus 1 near an NFC-enabled reader.

In some embodiments, the Eye2Tap apparatus 1 may be configured to interact with a medical smart hub computer system to provide remote monitoring of biometric data 120, including heart rate 420 and body temperature 430 (FIG. 11).

In some embodiments, the NFC module facilitates access control by enabling the user to unlock doors or vehicles 52 by tapping 35 the apparatus 1 near an NFC-enabled reader.

In some embodiments, the casing may be designed to be aesthetically integrated into the frame of the eyewear, ensuring that the apparatus 1 remains lightweight, ergonomic, and non-intrusive to the user (FIG. 4).

In some embodiments, the apparatus 1 may be configured to automatically adjust the settings of eyewear based on the detected biometric parameters, such as altering the lens tint in response to body temperature changes.

In some embodiments, the apparatus 1 may include a sensor module detects at least one biometric parameter selected from the group consisting of Glucose levels, PH, body temperature, heart rate, and motion data (FIG. 11).

In some embodiments, the memory module stores user-specific data, including medical records or eyeglass prescriptions. In some embodiments, the NFC module operates in a passive mode, deriving power exclusively from an external NFC reader during communication.

In some embodiments, the method may include providing an active power source in the casing, such as a rechargeable battery or capacitor, to power the apparatus 1 for extended functionality beyond NFC communication. In some embodiments, the NFC module 140 facilitates communication with external devices supporting Bluetooth Low Energy (BLE) or Wi-Fi for transmitting data to an external device.

In some embodiments, the method may include the step of storing the user's biometric data in a secure, encrypted format to ensure privacy during data transmission 36. In some embodiments, the apparatus 1 operates as a hybrid RFID/NFC device, using RFID for long-range communication and NFC for secure, short-range data transfer. In some embodiments, the apparatus 1 may be configured to store a log of biometric data and transmit the log to an external device upon request.

In some embodiments, the method includes securely storing the user's biometric data in an encrypted format to protect privacy during data transmission 37. The apparatus 1 may function as a hybrid RFID/NFC device, leveraging RFID for long-range communication and NFC for secure, short-range interactions. Additionally, the apparatus 1 may be configured to maintain a log of biometric data and transmit it to an external device upon request. In further embodiments, the apparatus 1 may serve as a storage medium for various types of data, including songs, videos, NFTs, or other digital content. This functionality allows the device to store and manage multimedia files or blockchain-based assets, enabling versatile data storage and transfer capabilities in conjunction with its biometric and communication features (FIG. 3).

In some embodiments, the Programmable Eyewear Accessory includes a shell composed of silicone, plastic, or rubber, designed to provide comfort and ensure that the eyewear remains securely positioned during use. The shell may wrap around the eyewear frame but is structured to maintain a distance, thereby preventing interference with the integrated NFC components and ensuring optimal communication (FIG. 7).

In some embodiments, the shell may incorporate programmable features, allowing for aesthetic customization such as LED lighting or decorative elements. These features may be controlled via an external device, such as a smartphone, enabling dynamic personalization (FIG. 4).

In some embodiments, the accessory integrates a hybrid RFID/NFC communication system, providing dual communication capabilities. The RFID component supports long-range interactions, while the NFC component facilitates secure, short-range data exchanges. This hybrid configuration ensures versatility in communication and enhances privacy for sensitive data transmissions.

In some embodiments, the device is configured to store biometric data securely in an encrypted format. This biometric functionality may support use cases such as user authentication, health monitoring, or access control. The accessory may log user-specific data and transmit it to external devices upon request, ensuring secure and traceable data management 12.

In some embodiments, an external device functions as a data retrieval system through apparatus 1, facilitating the extraction of data from multimedia storage mediums. This capability allows users to efficiently store, organize, and manage various digital files, including music, videos, and other types of multimedia content. Furthermore, the apparatus coupled to a device is designed to support blockchain-based digital assets, such as Non-Fungible Tokens (NFTs), providing features for secure storage and seamless transfer of these assets. This ensures a reliable solution for managing both traditional digital media and emerging blockchain-powered collectibles or assets.

In some embodiments, the accessory apparatus 1 includes a data logging module to maintain a record of user activity, biometric readings, or multimedia usage. This data may be transmitted to external devices, such as smartphones or cloud storage systems, for further analysis, backup, or sharing.

In some embodiments, the shell may incorporate various attachment mechanisms, such as magnetic fasteners, snap-on clips, adhesive strips, or cavity-based slip-fit designs, ensuring compatibility with different eyewear frame styles (FIG. 10).

In some embodiments, the apparatus 1 is designed to function as an environmental sensor, capturing data such as UV exposure, temperature, or humidity 140. This data may be used to enhance user comfort or provide health-related insights.

In some embodiment the accessory Advantages The disclosed invention offers a versatile apparatus 1 for eyewear that: Improves comfort through its ergonomic shell design. Provides programmable features for customization and aesthetic enhancement. Ensures secure data management through encrypted storage and hybrid communication technologies. Enables multimedia and blockchain asset management for diverse applications. Facilitates biometric data logging and health monitoring for advanced user interactions.

In some embodiments, the casing may be attached to the temple arm or bridge of the eyewear frame using a clip-on, magnetic, or adhesive coupling mechanism. In some embodiments, the apparatus 1 detects an abnormal biometric reading, such as an elevated heart rate or body temperature, and sends a real-time alert to an external device. In some embodiments, the method may include the step of synchronizing the stored biometric data with a remote healthcare system for remote monitoring of the user's health status.

In some embodiments, the NFC module facilitates access control by enabling the user to unlock doors or vehicles by tapping 35 the apparatus 1 near an NFC-enabled reader. In some embodiments, the method may include the step of automatically adjusting the settings of eyewear based on the detected biometric parameters, such as adjusting lens tint based on body temperature changes.

In some embodiments, the apparatus 1 may be used to facilitate secure, contactless payments by storing virtual payment card credentials on the NFC module. In some embodiments, the method may include providing the user with notifications via an external device when their biometric parameters exceed predefined thresholds. In some embodiments, the sensor module may be designed to make contact with the skin of the user for accurate biometric readings during normal wear.

In some embodiments, the method may include configuring the apparatus 1 to communicate with a smart home system, adjusting environmental settings such as temperature and lighting based on the user's biometric data. In some embodiments, the method may include enabling the apparatus 1 to interact with external devices for health tracking and wellness management, such as fitness trackers or medical devices. In some embodiments, the apparatus 1 transmits data to an external device only when the NFC module detects a valid user ID or authentication token to ensure secure communication.

In some embodiments, the apparatus may also include a method for integrating a wearable device with eyewear frames, including attaching a casing containing a sensor module, a memory module, and an NFC module to the eyewear frames. Embodiments may also include configuring the sensor module to detect one or more biometric parameters from a user. Embodiments may also include storing the detected biometric parameters and/or user-specific data in the memory module. Embodiments may also include transmitting the stored data to an external processor via the NFC module.

In some embodiments, the present invention, known as the Eye2Tap Programmable Eyewear Accessory, tackles a wide range of challenges in healthcare, personal convenience, and data management. By incorporating advanced technologies such as Near Field Communication (NFC), Radio Frequency Identification (RFID), biometric sensors, and ergonomic design, this innovative device creates a versatile solution adaptable to different markets and applications. The invention specifically addresses issues related to data management for eyewear users, comfort, biometric monitoring, digital economy integration, compatibility, environmental concerns, privacy, and aesthetic appeal.

In some embodiments, a significant challenge faced by eyewear users involves managing essential information, such as prescriptions, medication instructions, or identification details. Traditional eyewear lacks the capability to store or access such information, resulting in inefficiencies. The present invention, referred to herein as the Eye2Tap Adapter, integrates NFC-enabled modules to facilitate digital prescription storage, recovery of lost eyewear, and digital reminders for medication administration, thereby enhancing convenience and workflow efficiency in optometry clinics.

In some embodiments, another common problem with traditional eyewear is the discomfort and instability experienced during extended periods of wear or high-mobility activities. The Eye2Tap Adapter addresses this issue through ergonomic design and the use of lightweight materials such as silicone and flexible polymers. The adapter incorporates comfort-optimized components to minimize pressure points and features secure attachment mechanisms. These enhancements provide stability and comfort, particularly beneficial to athletes and everyday users.

In some embodiments, the present invention further addresses the lack of integrated biometric monitoring capabilities, which have become increasingly important due to the prevalence of chronic health conditions. By embedding biometric sensors in discreet locations on the eyewear, the Eye2Tap Adapter enables real-time monitoring of biometric parameters, such as heart rate, body temperature, and oxygen saturation, thereby facilitating health management for users with chronic illnesses.

In some embodiments, modern eyewear generally lacks digital functionality for tasks such as payments, access control, or loyalty programs. The Eye2Tap Adapter addresses this shortcoming by incorporating NFC and RFID functionalities, enabling users to make contactless payments, store loyalty rewards, and use their eyewear as a digital key for access control. This advancement enhances the utility of eyewear within the digital economy and streamlines interactions for users.

In some embodiments, to cater to the diverse needs of various eyewear styles and markets, the Eye2Tap Adapter is offered in multiple tailored designs. For healthcare, the adapter includes biometric-enabled features for health monitoring; for retail environments, it provides simplified NFC modules for payments and loyalty rewards; and for the luxury market, it incorporates customizable aesthetic enhancements. These variations accommodate the specific needs of different user groups, including athletes and fashion-conscious individuals.

In some embodiments, the Eye2Tap Adapter also addresses energy constraints and environmental concerns by incorporating energy-efficient features. The adapter utilizes energy harvesting technologies, such as solar and kinetic modules, in addition to wireless charging capabilities, to provide sustainable power solutions. These features minimize the need for bulky components while extending the battery life of the device.

In some embodiments, privacy and security are critical components for ensuring user trust, especially within digital healthcare and payment systems. The Eye2Tap Adapter implements data security measures, including encrypted storage, user authentication, and secure communication standards. These measures ensure reliable data sharing between healthcare providers and users, while maintaining confidentiality.

In some embodiments, aesthetic appeal is also a key focus of the present invention, as many functional accessories tend to overlook style. The Eye2Tap Adapter offers customizable design elements, such as programmable LED lighting, decorative shells, and a range of color and material options. These features allow the seamless integration of both function and fashion, thereby appealing to style-conscious users.

In some embodiments, the Eye2Tap Programmable Eyewear Accessory revolutionizes the traditional concept of eyewear by transforming it into a multifunctional platform capable of meeting a broad spectrum of user needs. With its modular design and tailored variations, the adapter is suitable for use in healthcare, retail, athletic, and luxury markets, effectively solving challenges related to comfort, utility, health monitoring, and data security, while also incorporating style and sustainability.

What is claimed is:

1. A programmable eyewear accessory apparatus comprising:
a shell configured to attach to eyewear frames while maintaining a predetermined distance between said frames and internal components;
a sensor module positioned within said shell;
a power management system; a memory module;
an antenna positioned for signal transmission; and
wherein said shell comprises securing points for stable attachment to said eyewear frames.

2. The apparatus of claim 1, wherein said shell comprises biocompatible shape-memory polymers.

3. The apparatus of claim 1, wherein said attachment mechanism comprises at least one of: magnetic coupling, mechanical locking, adhesive bonding, cavity-based fitting.

4. The apparatus of claim 1, wherein said biometric sensor array monitors at least one selected from the group consisting of heart rate, body temperature, glucose levels, pH, oxygen saturation, and motion.

5. The apparatus of claim 1, wherein said memory module stores one or more selected from the group consisting of medical records, prescriptions, vehicle settings, payment credentials, and digital assets.

6. An eyewear prescription data collecting apparatus comprising:
a shell configured as an attachment mechanism to accessory of an eyewear frame;
at least one NFC module positioned at a predetermined distance from said frame;
a mounting mechanism for securing to eyewear frames; and
an interference minimization system; wherein said apparatus enables data management and communication functionality.

7. The apparatus of claim 6, wherein said shell comprises at least one of: biocompatible materials, ergonomic design, adjustable components, glow in the dark capabilities.

8. The apparatus of claim 6, wherein said mounting mechanism comprises at least one of: Frame-specific attachment points, Stability enhancement features and a user comfort element.

9. The apparatus of claim 6, wherein said interference minimization system comprises at least one of: material shielding, distance optimization, signal protection.

10. The apparatus of claim 6, further storing at least one of: medical records, NFT data, user preferences and biometric information.

11. The apparatus of claim 6, wherein said NFC module comprises at least one of: an external device scanning capability, multiple chip compatibility, secure data transmission, variable functionality levels.

12. A method of enhancing eyewear functionality comprising at least one of:
attaching an enhancement module to eyewear, biometric data,
processing collected data, transmitting data, and managing power.

13. The method of claim 12, wherein attaching mechanism comprises at least one of: magnetic coupling, mechanical locking, adhesive bonding, and cavity-based fitting.

14. The method of claim 12, wherein accessing biometric data comprises at least one of: continuous heart rate monitoring, temperature sensing, glucose measurement, PH detection, oxygen saturation tracking, motion detection, and magnetic field detection.

15. The method of claim 12, wherein processing comprises at least one of: data encryption, real-time analysis, error correction, blockchain verification.

16. The method of claim 12, wherein transmitting comprises at least one of: NFC communication, RFID transmission, security protocols, and multi-factor authentication.

17. The method of claim 12, wherein managing power comprises:
an NFC energy harvesting.

18. The method of claim 12, further comprising at least one of: Payment processing, Access control and asset management.

19. The method of claim 12, further comprising at least one of: remote health monitoring, telemedicine integration, environmental sensing, smart hub interactions, smart home interaction and smart vehicle interaction.

20. An eyewear enhancement apparatus comprising:
a housing configured for attachment to an eyewear frame, said housing comprising an internal cavity;
an NFC chip disposed within said internal cavity;
a memory module operatively coupled to said NFC chip;
wherein said internal cavity positions said NFC chip at a predetermined distance from said eyewear frame to maintain communication functionality by minimizing interference.
